(19) United States
(12) Patent Application Publication
(10) Pub. No.: US 2008/0142003 A1
(43) Pub. Date: Jun. 19, 2008

(54) MRI Compatible Airway Management Device

(75) Inventor: William A. Depel, Merrillville, IN (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: ARCADIA MEDICAL CORPORATION, Schaumburg, IL (US)

(21) Appl. No.: 11/618,851

(22) Filed: Dec. 31, 2006

Related U.S. Application Data
(63) Continuation-in-part of application No. 11/610,359, filed on Dec. 13, 2006, now abandoned.

(51) Int. Cl. A61M 15/00

(52) U.S. Cl. 128/200.24

(57) ABSTRACT

An airway management device comprises an elongated tube having a distal end and a proximal end, the distal end used to establish an airway for a patient. At least a portion of the elongated tube is reinforced with reinforcing means that is compatible with Magnetic Resonance Imaging (MRI) procedures. The reinforcing means may comprise, for example, titanium, a titanium alloy, or tungsten, and may be in a helical coil or other configurations.

### CROSS REFERENCE TO RELATED APPLICATIONS

This application is a continuation-in-part of U.S. application Ser. No. 11/610,359, filed Dec. 13, 2006, the entirety of which is hereby incorporated by reference.

### BACKGROUND OF THE INVENTION

1. Field of the Invention

The present invention relates to airway management devices such as tubes used to assist in patient breathing.

2. Description of the Related Art

As a result of injury or disease, an individual's lungs may become too weak to sustain a sufficient flow of oxygen to the body as well as to remove carbon dioxide from the lungs. Under these circumstances it may be necessary to aid the lungs through forms of mechanical assistance, such as in mechanical ventilation. In one exemplary form, mechanical ventilation involves the introduction of a tracheal tube into an airway of the individual. For example, an endotracheal tube may be inserted through the individual's mouth or nose and into the trachea. Alternatively, a tracheostomy tube may be inserted through an incision or opening in the individual's neck and into the trachea. Typically, such tubes include a main body portion in the form of an elongated tube made of flexible material, such as polyvinylchloride. Alternatively, the tubes may be rigid, made of a material such as stainless steel or a stiff plastic. Many other types of airway management devices exist including, but not limited to, nasal tubes, epistaxis catheters, oral tubes, endobronchial tubes, laryngeal masks, pharyngeal masks, tracheal stents, and bronchial stents.

In operation, the distal end of the tube is positioned to rest at or slightly above the carina of the lungs. A well-humidified oxygen/air mixture is then introduced through the proximal end of the tube to provide oxygen to the lungs. Under less severe circumstances, the oxygen/air mixture can be supplied through the tube using continuous positive airway pressure (CPAP). When CPAP is used, the individual uses his or her own lung power to exhale the expired gas. However, under more severe circumstances, it is necessary to use mechanically controlled ventilation with a positive end expiratory pressure (PEEP).

Because endotracheal and tracheostomy tubes are commonly formed from a flexible material, they are subject to being “pinched” or “kinked” such that gas cannot be supplied to nor removed from the individual's lungs. For example, once a flexible tracheostomy tube is inserted through an incision in the individual's neck, subsequent rotation of the individual's head to the left or right may cause the central opening in the tube to pinch shut due to the pliable nature of the tube material. In order to help alleviate this problem, some tracheal tubes have been reinforced with stainless steel wire, thus making the tubes “kink resistant.”

One of the main drawbacks of tracheal tubes reinforced with a metal such as stainless steel is their incompatibility with Magnetic Resonance Imaging (MRI) devices. The use of MRI is becoming more common, and imaging with this modality is of increasing importance. Current medical devices, such as airway management devices, made of stainless steel or similar materials produce a large MR imaging artifact. Thus, diagnostics with MRI are more limited because of the image distortion produced near the airway management devices. Alloys that are radiopaque and MRI compatible are not common, but the ability to use materials with such characteristics is highly desirable.

Accordingly, there is a need for an airway management device having a tubular member for insertion into a patient's airway that is kink resistant and MRI compatible.

### BRIEF SUMMARY OF THE INVENTION

The present invention solves the foregoing problems by providing an airway management device comprising an elongated tube having a distal end and a proximal end, the distal end used to establish an airway for a patient. At least a portion of the elongated tube is reinforced with reinforcing means that is compatible with Magnetic Resonance Imaging (MRI) procedures. The reinforcing means may comprise, for example, titanium, a titanium alloy, or tungsten, and may be in a helical coil or other configurations.

In another aspect of the present invention, the airway management device may include an adjustable neck flange for positioning the distal end of the elongated tube at a desired location within the airway, and a neck flange locking device coupled to the neck flange for allowing axial adjustment of the neck flange between the proximal and distal ends of the elongated tube. The neck flange locking device comprises a threaded hub slidable along an outer surface of the elongated tube, a threaded nut slidable along the outer surface of the elongated tube and configured to receive the threaded hub, and a locking ring slidable along the outer surface of the elongated tube and disposed between the threaded nut and threaded hub. When the threaded hub is coupled to the threaded nut, the locking ring compresses against the outer surface of the elongated tube, thereby securing the neck flange at a desired axial position along the elongated tube.

### BRIEF DESCRIPTION OF THE DRAWINGS

FIG. 1 is a side view of a tracheostomy device, which is a first type of airway management device according to the present invention.

FIG. 2 is a cross section of a portion of the tracheostomy device of FIG. 1.

FIG. 3 is an exploded view of a neck flange locking device attachable to the tracheostomy device.

FIG. 4A is a side view of an alternative embodiment of a tracheostomy device according to the present invention with an elongated tube having a bend.

FIG. 4B is a side view of another alternative embodiment of a tracheostomy device according to the present invention with an elongated tube having a bend.

FIG. 5A is a side view of an alternative embodiment of a tracheostomy device according to the present invention having an extended connect feature.

FIG. 5B is a side view of another alternative embodiment of a tracheostomy device according to the present invention having a partially reinforced elongated tube.

FIG. 6 is a side view of an endotracheal device, which is a second type of airway management device according to the present invention.

FIG. 7 illustrates a section of the endotracheal device of FIG. 6 showing a tip portion.

FIG. 8 is a side view of an alternative embodiment of an endotracheal device according to the present invention.

FIG. 9 is a side view of another alternative embodiment of an endotracheal device according to the present invention.

FIG. 10 is a top view of a laryngeal mask device, which is a third type of airway management device according to the present invention.

### DETAILED DESCRIPTION OF THE INVENTION

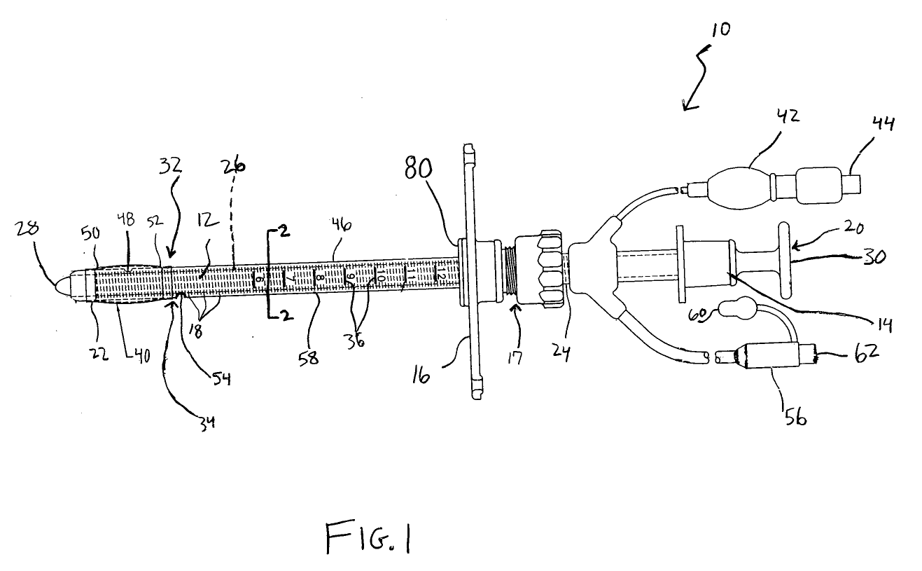

FIG. 1 is a side view of tracheostomy device 10, which is a first type of airway management device according to the present invention. Tracheostomy device 10 is configured to be inserted into a tracheostomy stoma, and generally includes elongated tube 12, connector 14, neck flange 16, neck flange locking device 17, tube reinforcement 18, and obturator 20. Elongated tube 12 includes distal end 22 and proximal end 24. Obturator 20 includes body 26 (shown in phantom lines), tip 28, and handle 30. As will be discussed in more detail to follow, tracheostomy device 10 may optionally include cuff system 32 and suction system 34. Tracheostomy devices may be formed from many materials, including but not limited to metals, plastics, and polymers. Thus, in one exemplary embodiment, elongated tube 12 may be formed from a soft silicone or polyvinylchloride (PVC) material, making it a versatile tube due to its ability to bend and flex as necessary to adapt to the specific contour of a patient's trachea.

In operation, an incision is first made in the patient's trachea by a clinician. Then, the clinician inserts tip 28 of obturator 20 into the incision, which acts as a guide for elongated tube 12 during the insertion process. The clinician typically applies a force on handle 30 of obturator 20 so as to gradually slide elongated tube 12 into the patient's trachea until distal end 22 is positioned at the desired location within the trachea. With tracheosomy devices having fixed neck flanges, this is typically determined by the position of distal end 22 when neck flange 16 contacts the patient's neck. However, as will be discussed in more detail to follow, adjustable neck flanges allow the clinician to adjust the length of elongated tube 12 that is inserted into the trachea. Finally, the clinician withdraws obturator 20 from elongated tube 12 by applying a slight force on neck flange 16 and gently pulling handle 30 until tip 28 is removed from proximal end 24, and secures neck flange 16 to the patient's neck with a neck strap (not shown in FIG. 1). Thus, obturator 20 is used only to guide elongated tube 12 during insertion and may be withdrawn immediately after elongated tube 12 is properly positioned within the trachea.

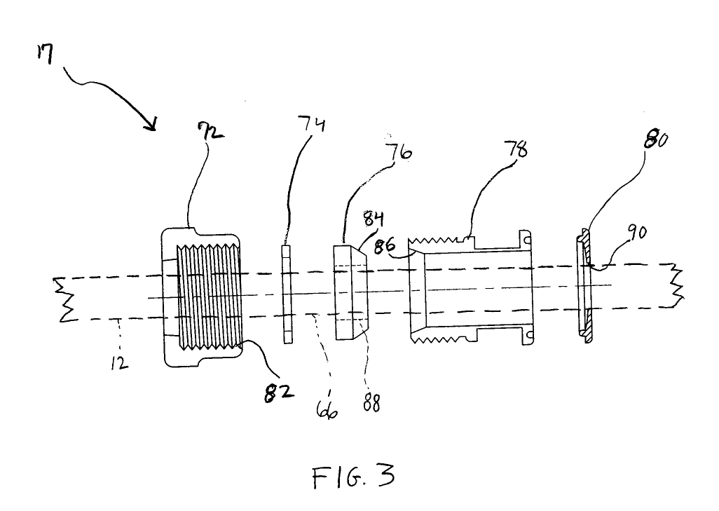

In the embodiment shown in FIG. 1, neck flange 16 is coupled to adjustable neck flange locking device 17. Neck flange locking device 17 allows neck flange 16 to slide axially along elongated tube 12 between distal end 22 and proximal end 24 and be locked into a desired position in order to obtain a proper fit within the patient's trachea. For example, neck flange 16 may be moved closer toward distal end 22 for patients having varied anatomies. Thus, tracheostomy device 10 is universal and reduces the need to carry these devices in various lengths depending on the anatomy of the patients. The outer surface of elongated tube 12 includes neck flange position markers 36, which serve as a visual indication of the position of neck flange 16 relative to distal end 22 of elongated tube 12. The specific details and components of locking device 17 will be discussed in further detail in reference to FIG. 3. Furthermore, although tracheostomy device 10 is shown as including an adjustable neck flange 16, embodiments wherein the neck flange is not adjustable are also contemplated. Thus, neck flange 16 is shown as adjustable merely for purposes of example, but may be rigidly fixed to elongated tube 12 in other embodiments.

Because elongated tube 12 is formed from a flexible material, it is inherently susceptible to being pinched or kinked as a bending or twisting force is applied to it. As an example, assume tracheostomy device 10 is inserted into the patient's trachea. Thereafter, turning or otherwise moving the patient's head with the tracheostomy device inserted within the trachea creates a risk of pinching or kinking elongated tube 12, thereby disrupting the airway provided by the tube. As one in the art would appreciate, allowing elongated tube 12 to be kinked or pinched is not acceptable due to the severe consequences that may result from an airway blockage. Thus, in order to make elongated tube 12 “kink resistant,” the walls of elongated tube 12 are reinforced with tube reinforcement 18. In the embodiment shown in FIG. 1, tube reinforcement 18 comprises a thin metallic helical wire coil formed integral with the walls of elongated tube 12. Most metals are ferromagnetic and good conductors of heat. These properties present potential safety risks, making airway management devices containing such metals non-compatible with Magnetic Resonance Imaging (MRI) procedures. In particular, the interaction of the magnetic and radiofrequency fields with such metals can lead to many undesirable consequences, including but not limited to trauma due to magnetic induction and thermal injury from radiofrequency induction heating of the metal.

In accordance with the present invention, airway management devices such as tracheostomy device 10 may include tube reinforcement 18 formed from materials such as, for example, titanium, a titanium alloy, or tungsten, which are all MRI compatible and have sufficient strength to provide resistance to kinking of elongated tube 12. For example, tube reinforcement 18 may be formed from Nitinol, which is a nickel-titanium shape-memory alloy. Titanium is a metallic element that is well-known for its high-strength to weight ratio. In addition, titanium has a high melting point, which makes it useful as a refractory metal (i.e., a class of metal extraordinarily resistant to heat). In particular, since titanium is non-ferromagnetic, the image artifacts produced during an MRI scan are negligible. Furthermore, because of its resistance to heat, the risk of thermal injury caused by titanium or a titanium alloy is minimal. Thus, when formed from a material such as titanium, tube reinforcement 18 may be MRI compatible, allowing use or tracheostomy device 10 in a patient during an MRI procedure without significant risk of injury to the patient. It is important to note that tracheostomy device 10 is assumed to include no metallic elements except for tube reinforcement 18. However, if other components of tracheostomy device 10 are formed from a metallic material, those components would also need to be formed from an MRI compatible material, such as titanium, so that the entire airway management device is “MRI compatible.”

Although tube reinforcement 18 was described as a thin metallic helical wire coil formed integral with the walls of elongated tube 12, the coil may alternatively be formed separately from the walls and inserted into the tube during manufacture of tracheostomy device 10. Furthermore, although tube reinforcement 18 was shown and described in reference to a helical coil, other configurations capable of providing resistance to kinks are also contemplated and within the intended scope of the present invention, such as, for example, non-helical, scaffolding, and lattice configurations.

As shown in FIG. 1, tracheostomy device 10 is a single cannula device because it contains only one tube member (elongated tube 12). In contrast, tracheostomy devices with an inner cannula include a second, inner tube that is inserted into elongated tube 12 and locked in place after obturator 20 is removed. One benefit of having a second, inner tube is that it may be withdrawn periodically for cleaning. It should be understood that all types of tracheostomy devices, including both single and double cannula devices, are contemplated and within the intended scope of the present invention.

Cuff system 32 of tracheostomy device 10 includes cuff 40, cuff pilot balloon 42, inflation valve 44, and cuff lumen 46. Cuff 40 is expandable outwardly into engagement with the inner wall of the patient's trachea, and is configured to ensure a tracheal seal, both to prevent loss of administered gas and to prevent aspiration of body fluids. Cuff 40 may be made from a material such as silicone, latex rubber, or plastic, and is mounted on distal end 22 of elongated tube 12. Cuff lumen 46 extends between pilot balloon 42 and cuff 40, providing an air passageway to inflate the cuff. A syringe or other type of inflation pump (not shown in FIG. 1) is coupled to inflation valve 44 of pilot balloon 42, and is configured to pump air through cuff lumen 46 toward cuff 40. Cuff lumen 46 includes an aperture 48 that allows air pumped through the lumen to enter the enclosed area defined by an inner surface of cuff 40 and an outer surface elongated tube 12. Ends 50 and 52 of cuff 40 are preferably coupled to elongated tube 12 with a silicone adhesive, although any type of adhesive capable of creating a seal between ends 50 and 52 of cuff 40 and elongated tube 12 that is safe for placement within the body is within the scope of the present invention.

During insertion into the patient's trachea, cuff 40 is in an uninflated state, as shown in FIG. 1. Once elongated tube 12 is properly positioned within the trachea, cuff 40 is inflated like a balloon by feeding air into the cuff through cuff lumen 46 to expand cuff 40 into engagement with the inner wall of the trachea to provide a seal thereagainst. When it is time to remove tracheostomy device 10, the syringe or air pump is typically used to suction the air from, and thus deflate, cuff 40, allowing elongated tube 12 to be removed from the trachea.

Inflation valves such as inflation valve 44 that are generally used in airway management devices typically include metal components that are not MRI compatible. Therefore, these inflation valves interact with the magnetic and radiofrequency fields produced during an MRI procedure, leading to the undesirable and dangerous consequences discussed above. Thus, inflation valve 44 may be designed without metal components, or having metal components made out of an MRI compatible metal such as titanium or a titanium alloy, so that the valve will not pose any risks during MRI procedures.

Cuff 40 of tracheostomy device 10 has been described as an “air cuff.” However, an alternative type of cuff for a tracheostomy device embodies a cover filled with resilient material, such as compressible foam, with the cuff normally being disposed in an expanded position and being collapsed by applying a vacuum thereto during insertion or removal of the tube into or from the trachea, respectively. Another alternative type of cuff is similar to an air cuff, but is injected with a liquid, such as a saline solution, to expand the cuff from a normally deflated state.

Suction system 34 of tracheostomy device 10 includes suction hole 54, suction port 56, and suction lumen 58. Suction hole 54 is positioned proximal to end 52 of cuff 40, and is designed to suction away subglottic secretions above cuff 40 when positioned within the trachea. Because of its integrated design within tracheostomy device 10, suction system 34 avoids trauma to the patient's vocal cords that may occur during manual catheter suctioning above the cuff as is commonly performed with a separate device. Suction lumen 58 extends between suction hole 54 and suction port 56, providing a passageway for the secretions being suctioned. A vacuum device (not shown in FIG. 1) is coupled to suction port 56, and is configured to provide a source of suction with adequate vacuum to pull secretions from the cuff area into suction hole 54 and through suction lumen 58. Suction system 34 also includes suction port cover 60, which is shown in the disconnected position in FIG. 1, but which may be inserted into open end 62 of suction port 56 when the vacuum device is disconnected.

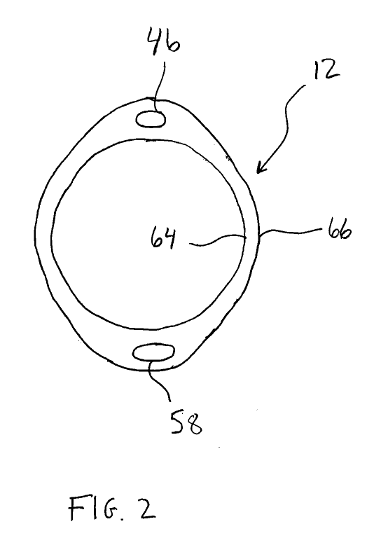

FIG. 2 is a cross section taken along line 2-2 of elongated tube 12 in FIG. 1. As shown in FIG. 2, cuff lumen 46 and suction lumen 58 are formed integral with elongated tube 12 during the manufacturing process. In particular, cuff and suction lumens 46 and 58 are disposed between inner wall 64 and outer wall 66 of elongated tube 12. However, in other embodiments, cuff and suction lumens 46 and 58 may be formed as separate tube members that are adhered to inner wall 64 or outer wall 66 of elongated tube 12. In addition, the lumens may be designed having any number of cross-sectional shapes, including but not limited to circular, oval, and D-shaped. Furthermore, although lumen 58 is depicted as having a larger cross-sectional area than lumen 46, these areas could vary in other embodiments.

FIG. 3 is an exploded view of neck flange locking device 17, which includes threaded nut 72, washer 74, locking ring 76, threaded hub 78, and wiper seal 80. In FIG. 3, elongated tube 12 is shown in phantom lines in order to depict the position of neck flange locking device 17 relative to the tube. When assembled, washer 74 is inserted into threaded portion 82 of threaded nut 72. Then, tapered end 84 of locking ring 76 is inserted into threaded hub 78, which has a mating surface 86 for mating with tapered end 84. Next, threaded hub 78 is threaded into threaded nut 72, thereby sandwiching locking ring 76 between washer 74 and threaded hub 78. Locking ring 76 may be made from a flexible material such as silicone. Thus, As threaded hub 78 is threaded into threaded nut 72, flexible locking ring 76 is compressed, resulting in deformation of the ring, which causes inner wall 88 of locking ring 76 to contact outer wall 66 of elongated tube 12. Once hub 78 is threaded into nut 72 by some set amount, locking ring 76 will transfer a sufficient force onto outer wall 66 of elongated tube 12 such that neck flange locking device 17 will no longer be able to slide axially relative to elongated tube 12. Locking ring 76 may be designed so that when it is compressed, it distributes a substantially uniform force on elongated tube 12. As one of skill in the art would appreciate, neck flange locking device 17 of the present invention allows a user to reposition and “lock” neck flange 16 at any location along the axial length of elongated tube 12.

Wiper seal 80 is attached to threaded hub 78 with an adhesive, such as a silicone adhesive or other suitable adhesive, and is designed such that inner edge 90 forms a seal with outer wall 66 of elongated tube 12. This seal prevents liquids such as bodily fluids or mucus from entering neck flange locking device 17 and creating a build-up of moisture.

Locking ring 76 and wiper seal 80 may be formed from any number of pliant materials such as, for example, silicone. Washer 74 is preferably formed from any number of materials such as a plastic or Teflon material. Finally, threaded nut 72 and threaded hub 78 may be formed from a rigid plastic material. However, it should be understood that these materials are listed for purposes of example and not for limitation, and that other materials having similar characteristics are also contemplated.

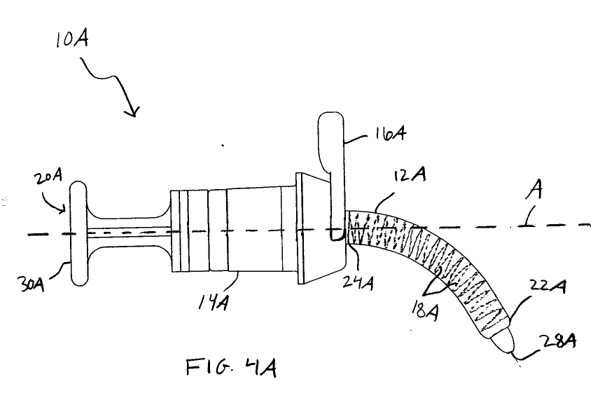

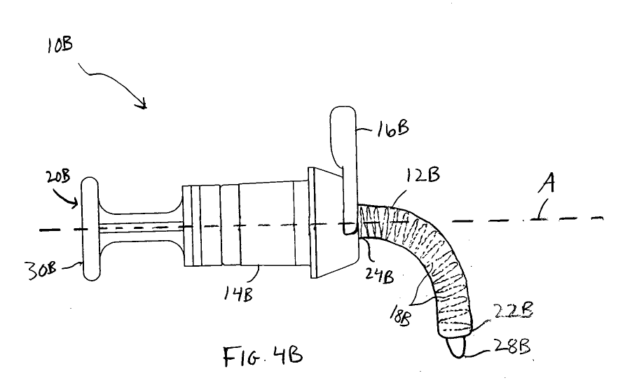

Although FIG. 1 illustrates a tracheostomy device having an elongated tube that is generally straight, other configurations are also possible. In particular, FIGS. 4A and 4B illustrate tracheostomy devices that include elongated tubes having a “bend.” Tracheostomy device 10A of FIG. 4A includes an elongated tube 12A having a bend of about 45 degrees relative to axis A. Similarly, tracheostomy device 10B of FIG. 4B includes an elongated tube 12B having a bend of about 90 degrees relative to axis A. Thus, one skilled in the art would appreciate that tracheostomy devices having elongated tubes with various other angular bends preferably within a range between about 45 degrees and about 90 degrees are also possible.

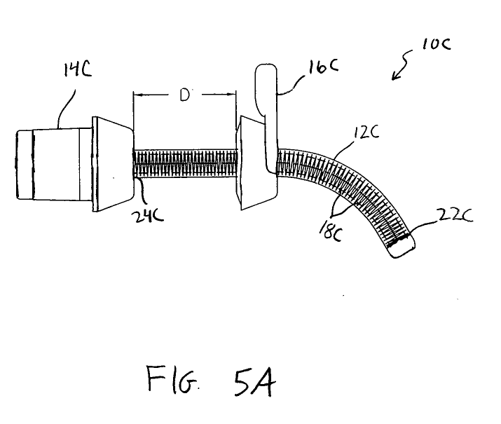

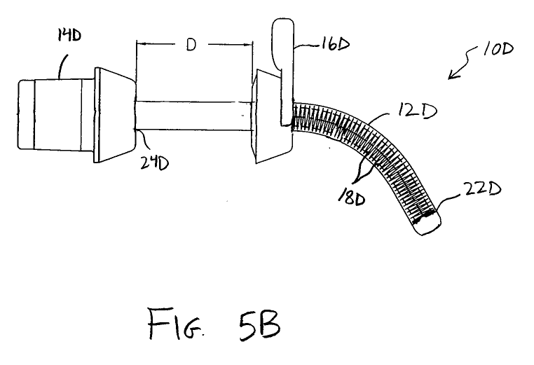

FIG. 5A is a side view of tracheostomy device 10C, which is another alternative embodiment of an airway management device according to the present invention. As shown in FIG. 5A, neck flange 16C is coupled to elongated tube 12C and spaced apart from connector 14C by a distance D. This type of design may be beneficial, for example, in pediatric and neonatal tracheostomy devices where it is necessary to move connector 14C away from the pediatric or neonatal patient's neck due to the limited space available for making connections to the device. As shown in FIG. 5A, tube reinforcement 18C is a helical coil formed from an MRI compatible material such as titanium or a titanium alloy, and extends from distal end 22C to proximal end 24C of elongated tube 12C. Thus, both the portion of elongated tube 12C that is distal to neck flange 16C (i.e., the portion that is within the patient's trachea) and the portion that is proximal to neck flange 16C (i.e., the portion that is outside of the patient's body) is kink resistant.

Although the embodiments of an airway management device described above have included tube reinforcement along substantially the entire length of the elongated tube, embodiments that include only partially reinforced tubes are also possible. For example, FIG. 5B shows tracheostomy device 10D, which is similar to tracheostomy device 10C described in reference to FIG. 5A. However, elongated tube 12D includes tube reinforcement 18D within only a portion of the tube. In particular, the portion of elongated tube 12D between the distal side of neck flange 16D and distal end 22D includes tube reinforcement 18D, while the portion of elongated tube 12D between the proximal side of neck flange 16D and proximal end 24D (distance D) includes no reinforcement. As a result, only the portion of elongated tube 12D that is inserted within the patient's trachea is kink resistant. Thus, one skilled in the art would appreciate that airway management devices in accordance with the present invention may be designed that have fully or partially reinforced elongated tube members.

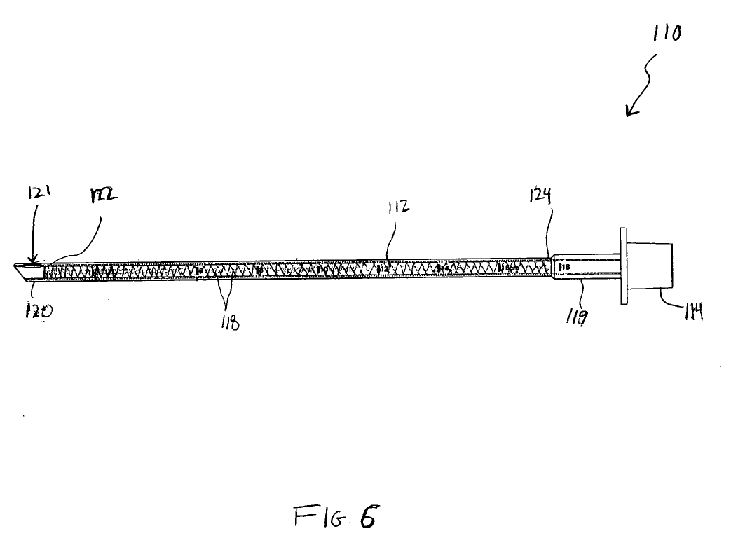

FIG. 6 is a side view of endotracheal device 110, which is a second type of airway management device according to the present invention. An endotracheal device is typically used in anaesthesia, intensive care, and emergency medicine for airway management and mechanical ventilation. Endotracheal device 110 is designed to be inserted into a patient's trachea, generally through the patient's mouth, but sometimes through the nares of the nose or through a tracheostomy stoma. As shown in FIG. 5, endotracheal device 110 generally includes elongated tube 112, connector 114, tube reinforcement 118, connector strain relief sleeve 119, and tip portion 120 having murphy eye 121. Connector strain relief sleeve 119 is designed to prevent kinking of the device near the junction of elongated tube 112 and connector 114. Elongated tube 112 includes distal end 122 and proximal end 124. Similar to tracheostomy tubes, endotracheal tubes may be formed from many materials, but are commonly formed from a flexible silicone or PVC material, which allows elongated tube 112 to flex and bend as it is being inserted into the patient's trachea. In addition, the flexible material allows elongated tube 112 to conform to changes in patient position after the tube is in place within the trachea. Although materials such as silicone or PVC are contemplated, one skilled in the art would appreciate that elongated tube 112 may be formed from numerous other flexible materials without departing from the intended scope of the present invention.

Endotracheal device 110 is reinforced with tube reinforcement 118 in order to make elongated tube 112 “kink resistant” as discussed above in reference to tracheostomy device 10. Once again, tube reinforcement 118 comprises a thin metallic helical wire coil formed integral with the walls of elongated tube 112, and is formed from an MRI compatible material as discussed above. As a result, endotracheal device 110 is both kink resistant and MRI compatible, allowing the device to be used during procedures involving MRI.

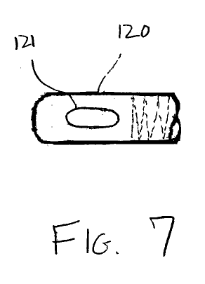

FIG. 7 illustrates a section of endotracheal device 110 showing tip portion 120. Tip portion 120 includes murphy eye 121, which is designed to prevent complete respiratory obstruction of tip portion 120 in the event that the open tip portion 120 were to become blocked by contact with the tracheal wall of the patient or occluded by, for example, mucus.

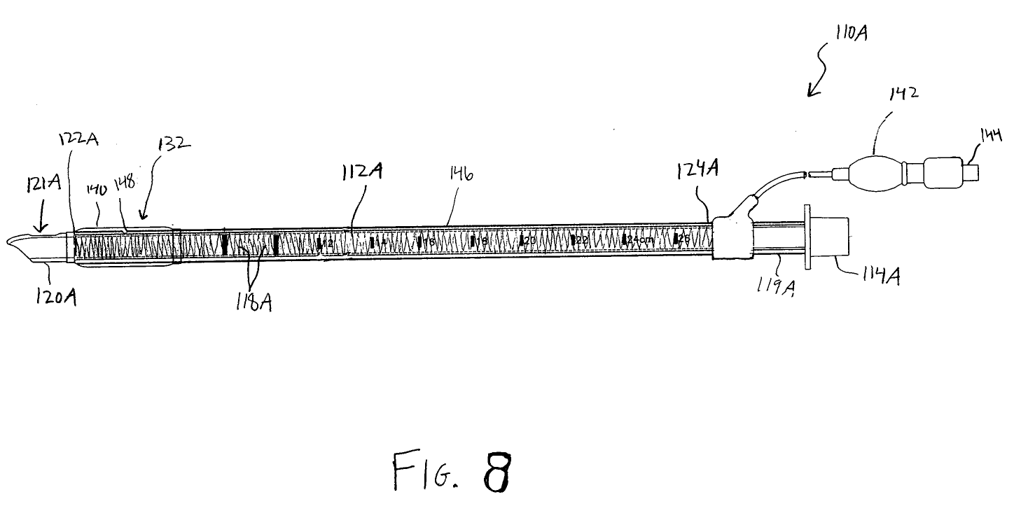

FIG. 8 is a side view of endotracheal device 110A, which is a first alternative embodiment of endotracheal device 110 according to the present invention, and wherein similar elements are given similar reference numerals. In particular, endotracheal device 110A is similar to endotracheal device 110, but further includes cuff system 132. Cuff system 132 is similar in both structure and operation to cuff system 32 described above in reference to tracheostomy device 10 in FIG. 1, and includes cuff 140, cuff pilot balloon 142, inflation valve 144, and cuff lumen 146. Other than the fact that cuff systems 32 and 132 have been incorporated into different devices, the systems themselves are the same. Therefore, the discussion above regarding the operation of cuff system 32 applies in the same manner to cuff system 132. Furthermore, as discussed in reference to cuff system 32, cuff system 132 may also utilize other types of cuffs, such as foam cuffs, without departing from the intended scope of the present invention.

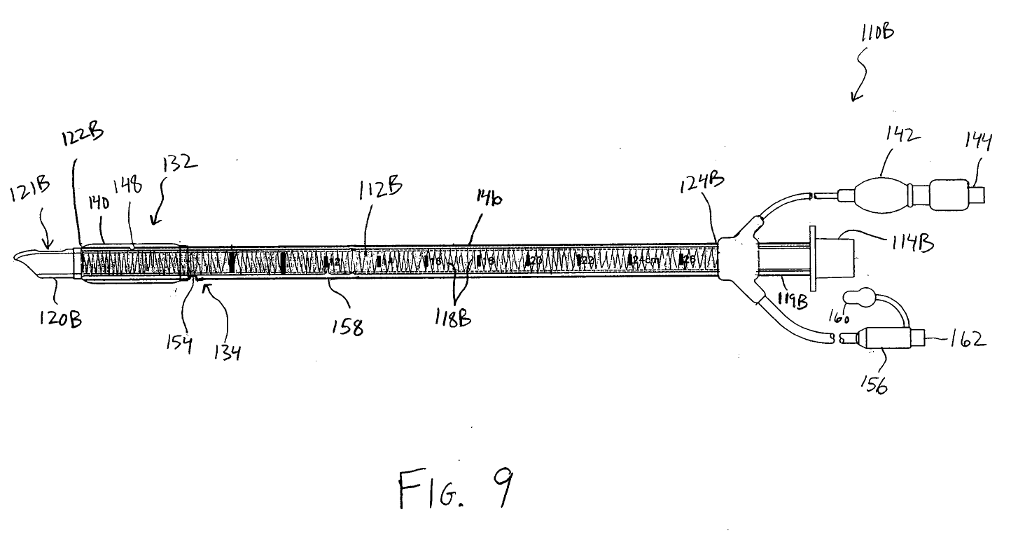

FIG. 9 is a side view of endotracheal device 110B, which is a second alternative embodiment of endotracheal device 110 according to the present invention, and wherein similar elements are given similar reference numerals. In particular, endotracheal device 110B is similar to endotracheal device 110A, but further includes suction system 134 designed to suction away subglottic secretions above cuff 140 when positioned within the trachea. Suction system 134 is similar in both structure and operation to suction system 34 described above in reference to tracheostomy device 10 in FIG. 1, and includes suction hole 154, suction port 156, and suction lumen 158. Other than the fact that suction systems 34 and 134 have been incorporated into different devices, the systems themselves are the same. Therefore, the discussion above regarding the operation of suction system 34 applies in the same manner to suction system 134.

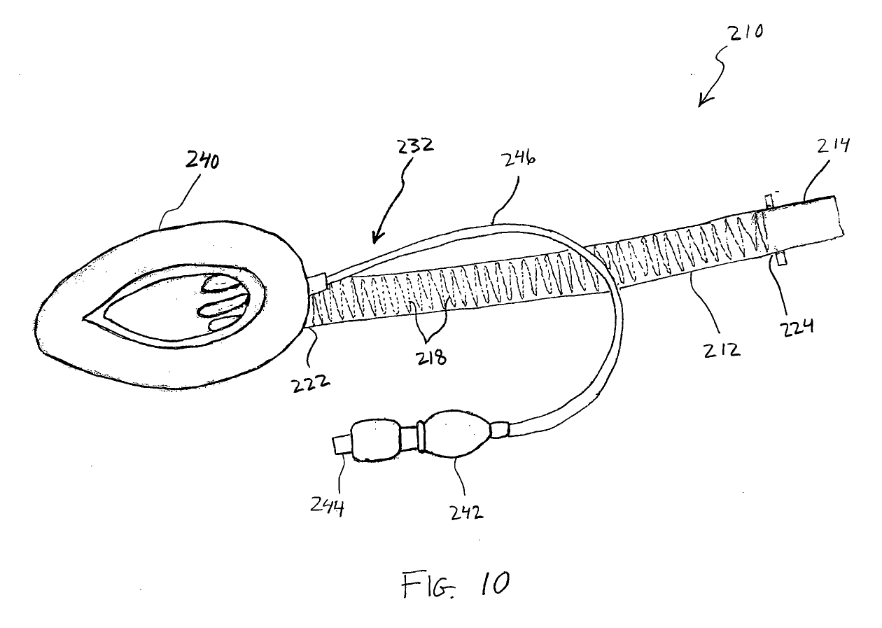

FIG. 10 is a top view of laryngeal mask device 210, which is a third type of airway management device according to the present invention. A laryngeal mask device is typically used in anaesthesia and in emergency medicine for airway management, and is designed to be inserted into a patient's pharynx. As shown in FIG. 10, laryngeal mask device 210 generally includes elongated tube 212, connector 214, tube reinforcement 218, and cuff system 232. Elongated tube 212 includes distal end 222 and proximal end 224. Similar to tracheostomy and endotracheal tubes, laryngeal masks may be formed from many materials, but are commonly formed from a flexible silicone or PVC material, which allows elongated tube 212 to flex and bend as it is being inserted into the patient.

Laryngeal mask device 210 is reinforced with tube reinforcement 218 in order to make elongated tube 212 “kink resistant.” Once again, tube reinforcement 218 is formed from an MRI compatible material as discussed above, thus creating a device that is both kink resistant and compatible for use during procedures involving MRI.

Cuff system 232 of laryngeal mask device 210 includes cuff 240, cuff pilot balloon 242, inflation valve 244, and cuff lumen 246. Cuff 240 is expandable outwardly after insertion into the patient's pharynx to create a seal. Cuff lumen 246 extends between pilot balloon 242 and cuff 240, providing an air passageway to inflate the cuff. A syringe or other type of inflation pump (not shown in FIG. 10) is coupled to inflation valve 244 of pilot balloon 242, and is configured to pump air through cuff lumen 246 to inflate cuff 240.

One skilled in the art would appreciate that although the present invention has been described in reference to tracheostomy, endotracheal, and laryngeal mask devices, numerous other airway management devices could benefit from an MRI compatible material for reinforcement within the device. For example, airway management devices including, but not limited to, pharyngeal masks, laryngeal tubes, pharyngeal tubes, perilaryngeal airways, and endobronchial tubes may be made both kink resistant and MRI compatible in accordance with the present invention. Thus, tracheostomy, endotracheal, and laryngeal mask devices are described for purposes of example and not for limitation.

Although the present invention has been described with reference to preferred embodiments, workers skilled in the art will recognize that changes may be made in form and detail without departing from the spirit and scope of the invention.

I claim:

1. An airway management device comprising:
an elongated tube having a distal end and a proximal end, the distal end used to establish an airway for a patient, wherein at least a portion of the elongated tube is reinforced with reinforcing means that is compatible with Magnetic Resonance Imaging (MRI) procedures.

2. The airway management device of claim 1, wherein the reinforcing means comprises titanium.

3. The airway management device of claim 2, wherein the reinforcing means is formed from a titanium alloy.

4. The airway management device of claim 1, wherein the reinforcing means comprises tungsten.

5. The airway management device of claim 1, wherein the reinforcing means is in a helical configuration.

6. The airway management device of claim 1, wherein the reinforcing means is in a non-helical configuration.

7. The airway management device of claim 1, wherein establishing an airway occurs by inserting the distal end of the elongated tube into an airway of the patient.

8. An airway management device comprising:
an elongated tube having a distal end and a proximal end, the distal end used to establish an airway for a patient, wherein at least a portion of the elongated tube is reinforced with an MRI compatible reinforcing means;
an expandable cuff surrounding the elongated tube near the distal end of the elongated tube; and
an opening in the elongated tube proximal to the expandable cuff to allow fluids in the airway to be suctioned away from an outer surface of the elongated tube proximal to the expandable cuff.

9. The airway management device of claim 8, wherein the elongated tube is formed from silicone.

10. The airway management device of claim 8, wherein the MRI compatible reinforcing means comprises titanium.

11. The airway management device of claim 10, wherein the MRI compatible reinforcing means is formed from a titanium alloy.

12. The airway management device of claim 8, wherein the MRI compatible reinforcing means comprises tungsten.

13. The airway management device of claim 8, wherein the reinforcing means is in a helical configuration.

14. The airway management device of claim 8, further comprising a tip portion at the distal end of the elongated tube having a main opening and a murphy eye, the murphy eye designed to prevent complete obstruction of the tip portion in the event that the main opening were to become blocked.

15. The airway management device of claim 8, wherein the elongated tube is reinforced with the MRI compatible reinforcing means from the distal end to the proximal end.

16. The airway management device of claim 8, further comprising a neck flange attachable to the elongated tube.

17. The airway management device of claim 16, wherein the elongated tube is reinforced with the MRI compatible reinforcing means from a distal side of the neck flange to the distal end of the elongated tube.

18. An airway management device comprising:
an elongated tube having a distal end and a proximal end, the distal end used to establish an airway for a patient;
an adjustable neck flange for positioning the distal end of the elongated tube at a desired location within the airway; and
a neck flange locking device coupled to the neck flange for allowing axial adjustment of the neck flange between the proximal and distal ends of the elongated tube, the neck flange locking device comprising:
a threaded hub slidable along an outer surface of the elongated tube;
a threaded nut slidable along the outer surface of the elongated tube and configured to receive the threaded hub; and
a locking ring slidable along the outer surface of the elongated tube and disposed between the threaded nut and threaded hub, wherein coupling the threaded hub to the threaded nut compresses the locking ring against the outer surface of the elongated tube to secure the neck flange at a desired axial position along the elongated tube.

19. The airway management device of claim 18, wherein the neck flange locking device further comprises a seal disposed between a distal end of the threaded hub and the outer surface of the elongated tube for preventing migration of liquids into the locking device.

20. The airway management device of claim 18, wherein at least a portion of the elongated tube is reinforced with reinforcing means.

21. The airway management device of claim 20, wherein the reinforcing means comprises titanium.

22. The airway management device of claim 21, wherein the reinforcing means is formed from a titanium alloy.

23. The airway management device of claim 20, wherein the reinforcing means comprises tungsten.

24. The airway management device of claim 18, further comprising an expandable cuff surrounding the elongated tube near the distal end of the elongated tube.

25. The airway management device of claim 24, further comprising a cuff lumen formed integral with the elongated tube and extending between the cuff and an inflation valve.

26. The airway management device of claim 25, wherein the cuff is an air inflatable cuff.

27. The airway management device of claim 25, wherein the cuff is a foam cuff.

28. The airway management device of claim 18, further comprising a suction opening in the elongated tube proximal to the cuff to allow fluids in the airway to be suctioned away from an outer surface of the elongated tube proximal to the cuff.

29. The airway management device of claim 28, further comprising a lumen formed integral with the elongated tube and extending between the suction opening and a suction port.

30. The airway management device of claim 18, further comprising insertion depth measurement markings on the outer surface of the elongated tube for determining depth of the distal end of the elongated tube within an airway of the patient.

31. The airway management device of claim 30, wherein the insertion depth measurement markings are located on one side of the elongated tube.

32. The airway management device of claim 30, wherein the insertion depth measurement markings are located on two sides of the elongated tube.

33. The airway management device of claim 18, wherein the airway management device comprises a tracheostomy device.

34. The airway management device of claim 18, wherein the airway management device comprises an endotracheal device.

35. An airway management device comprising:
an elongated tube having a distal end and a proximal end, the distal end used to establish an airway for a patient;
an adjustable neck flange for positioning the distal end of the elongated tube at a desired location within the airway; and
a neck flange locking device coupled to the neck flange for allowing axial adjustment of the neck flange between the proximal and distal ends of the elongated tube, the neck flange locking device comprising:
a threaded hub slidable along an outer surface of the elongated tube;
a threaded nut slidable along the outer surface of the elongated tube and configured to receive the threaded hub; and
a seal disposed between a distal end of the threaded hub and the outer surface of the elongated tube for preventing migration of liquids into the locking device.

36. The airway management device of claim 35, wherein the neck flange locking device further comprises a locking ring slidable along the outer surface of the elongated tube and disposed between the threaded nut and threaded hub, wherein coupling the threaded hub to the threaded nut compresses the locking ring against the outer surface of the elongated tube to secure the neck flange at a desired axial position along the elongated tube.
